(19) United States
(12) Patent Application Publication
(10) Pub. No.: US 2022/0015357 A1
(43) Pub. Date: Jan. 20, 2022

(54) AERIAL GERMICIDE FORMED FROM AN OPERATIVE FLUID AND METHOD OF USE FOR SAME

(71) Applicants: Steven Semoff, New City, NY (US); Spence Levy, Palmetto Bay, FL (US)

(72) Inventors: Steven Semoff, New City, NY (US); Spence Levy, Palmetto Bay, FL (US)

(21) Appl. No.: 17/175,208

(22) Filed: Feb. 12, 2021

Related U.S. Application Data
(60) Provisional application No. 63/053,028, filed on Jul. 17, 2020.
(60) Provisional application No. 63/077,178, filed on Sep. 11, 2020.

(51) Int. Cl. A01N 25/04; A01N 31/02; A61L 9/14

(52) U.S. Cl. CPC A01N 25/04; A61L 9/14; A01N 31/02

(57) ABSTRACT

An aerial sanitizer for use in indoor spaces may be created by dispersing an operative fluid within a fluid dispersion device or other like apparatus. The operative fluid may be comprised of a first compound, such as triethylene glycol, or any other like compound sufficiently effective as an aerial germicide. Because of the hygroscopicity and low toxicity of triethylene glycol, or other like germicidal compounds, the operative fluid may further include a second compound, such as deionized water, to devise the operative fluid in dynamic equilibrium with the surrounding ambient air, such that the triethylene glycol or other like compound will abstain from ambient water uptake, thereby preventing the dilution of the operative fluid. Such an operative fluid may further include a third compound, such as alternative essential oils, for both germicidal activity and the provision of certain sensory signals, whether organoleptic or otherwise.

### CLAIM OF PRIORITY

The present invention claims priority to a previously filed and currently pending Provisional patent application having Ser. No. 63/077,178 and a filing date of Sep. 11, 2020, which itself claims priority to a previously filed and currently pending Provisional patent application having Ser. No. 63/053,028 and a filing date of Jul. 17, 2020, each of which are hereby incorporated by reference in their entireties.

### BACKGROUND OF THE INVENTION

### Field of the Invention

The present invention is directed to an operative fluid for sanitizing air, an aerial germicide formed therefrom, and a method of dispersing such an aerial germicide to reduce and eliminate airborne microorganisms within an interior space.

### Description of the Related Art

Aerial sanitizers are sometimes used to reduce and mitigate the growth of airborne microorganisms. However, typical systems of aerial sanitization rely on the creation of aerosol formulations through aerosol canisters, which commonly require the use of propellants to pressurize such aerosol canisters to ensure the desired ingredients are properly released. Most common propellants are volatile hydrocarbons, including propane, n-butane, and isobutane, all of which may cause certain occupational and/or health risks through either acute and/or chronic exposure thereto.

One particular composition found to comprise sufficient toxicity to pathogens, thereby constituting an effective agent in killing airborne microorganisms through such aerial sanitization systems is triethylene glycol (“TEG”). However, although the germicidal nature of triethylene glycol has been known for some time, the efficacy of its use in such aerial sanitization systems has been found wanting as a result of inefficiencies stemming both from such aerial sanitization systems and the propensity of triethylene glycol to absorb water from ambient air due to its hygroscopic nature, thereby reducing its concentration and efficacy as an aerial germicide.

Accordingly, traditional uses of triethylene glycol in aerial sanitization systems comprise aerosol formulations disposed within the previously mentioned aerosol canisters. In so doing, the hygroscopic nature of triethylene glycol may be reduced, as water intake from ambient air is effectively prevented by disposing the triethylene glycol formulation within a closed system.

However, such traditional aerial sanitization systems have their own problems. For instance, because triethylene glycol quickly condenses on surfaces leading to a large reduction in the amount of triethylene glycol in the surrounding airspace and thereby reducing the efficacy of same as an aerial sanitizer after a certain amount of time, and because such aerosol canisters require user interaction, it may be understood the triethylene glycol formulation is typically not applied often enough to remain effective. Conversely, the triethylene glycol is typically used in a manner merely providing small area-of-effect uses, failing to both sufficiently fill and saturate the surrounding airspace. Additionally, when the aerosol droplet size of triethylene glycol is large enough, it is unable to bind to airborne pathogens and is thus ineffective as a sanitizer. In order to maintain the efficacy of triethylene glycol as a sanitizing agent, it is important to disperse the triethylene glycol in such a way to keep the droplet size small enough to bind to airborne pathogens.

Likewise, as stated previously, such aerosol canisters typically require hydrocarbon propellants to effectuate the expulsion of the triethylene glycol formulation therefrom. However, triethylene glycol has a low solubility in such hydrocarbon propellants and, accordingly, the efficacy of the triethylene glycol in such systems is reduced as an ideal mixture fails to form, thereby leading to inconsistent application of the triethylene glycol. Moreover, such hydrocarbon propellants are commonly considered volatile organic compounds which damage the environment and may have short and/or long-term adverse health effects when dispersed in an enclosed environment.

Conversely, alternative methods of aerial sanitization may comprise applying pressurized air to an aerosol formulation, thereby reducing the aerosol formulation into small particles before releasing the gaseous air and particle mixture. In so doing, the particles comprising the aerosol formulation may behave as a gas, thereby reaching all areas of a room and settling on surfaces and/or airborne pathogens, and disinfecting same. The mere use of triethylene glycol in such systems, such as the one disclosed in U.S. Pat. No. 10,583,449, however, raises its own issues.

Specifically, because the reservoir housing the triethylene glycol solution in such systems is not conventionally disposed in a closed system, or is otherwise exposed to ambient air as the volume of the triethylene glycol is used, the triethylene glycol solution will be exposed to ambient air. As previously discussed, the ambient air in such situations may dilute the triethylene glycol solution as a result of the condensation of water disposed therein in combination with the hygroscopic nature of triethylene glycol. Accordingly, such systems are often inefficient, as the triethylene glycol solution naturally dilutes over time, thereby continually losing its efficacy. Likewise, such systems also commonly suffer from failing to consistently provide a sufficient amount of triethylene glycol to the surrounding airspace to ensure same remains saturated therewith. As may be understood, such failure essentially renders the triethylene glycol obsolete, as airborne pathogens will always remain unless the surrounding airspace remains at a high concentration of triethylene glycol.

Accordingly, a solution to the foregoing problems should seek to reduce, or otherwise eliminate, the reliance on aerosol canisters for the application of triethylene glycol, or other like chemical compositions which may operate as an aerial germicide, due to the desire to reduce the use of such volatile hydrocarbon propellants. Such a solution should further be disposed to prevent the aforementioned ambient water uptake which has plagued such systems. Moreover, such a system should be disposed to automatically disperse the triethylene glycol at the appropriate droplet size, or other like composition, at predetermined intervals such that the surrounding airspace may remain saturated with an aerial germicide. Finally, such a solution should also incorporate certain other chemical compositions which may either further benefit the germicidal activity proffered by the triethylene glycol or other like composition, or otherwise provide a sensory signal to people disposed in the surrounding airspace, thereby indicating the surrounding airspace has been sanitized and is free of such airborne pathogens.

### SUMMARY OF THE INVENTION

The present invention is directed to a chemical composition, the product formed therefrom, and a method for producing same. Specifically, the present invention is directed to a fluid dispersion, which may comprise an aerial germicide, an aerial disinfectant, and/or any other like aerial sanitizer designed to be utilized in accordance with a fluid dispersion device. Hereinafter, the terms “sanitizer,” “sanitizers,” “sanitized,” “sanitizing,” and “sanitization” refer specifically to the removal, sterilization, decontamination, and/or neutralizing of microorganisms, and may encompass a plurality of terms including, without limitation, “germicide,” “germicidal,” “disinfectant,” “disinfecting,” and any of their other forms or other like terms pertaining to a chemical agent designed to destroy, eliminate, or otherwise nullify pathogens and other microorganisms.

A fluid dispersion device designed to be utilized in accordance with the fluid dispersion may take a number of forms. Such a fluid dispersion device may comprise, for instance, a jet nebulizer, a concentric tube pneumatic nebulizer, a diffuser, or any other device so disposed to create an aerosol compound comprising a gaseous medium with miniscule liquid particles suspended therein. For instance, one such fluid dispersion device in which the present invention may be effectively formed is disclosed in U.S. Pat. No. 10,583,449, which discloses a fluid dispersion assembly disposed to generate a fluid dispersion in air with a distribution of a particular liquid at a uniform particle size.

More specifically, such a fluid dispersion device may comprise at least a reservoir for holding the pertinent operative fluid, an elongated tube disposing the reservoir in fluid communication with a compressed air source and a mixing chamber, and an outlet. Through such components, it may be understood the application of the compressed air source on the elongated tube may withdraw the operative fluid from the reservoir, according to the venturi effect. Then, as the operative fluid passes through the compressed air, the operative fluid will be dispersed into miniscule droplets before mixing therewith. Finally, upon such mixing, the resulting mixture may be expelled from such a fluid dispersion device into the surrounding environment.

Generally speaking, the aerial germicide formed through such a fluid dispersion device may result from an operative fluid. Such an operative fluid may comprise a chemical composition including at least a first compound. In at least one embodiment of the present invention, such a first compound may comprise a glycol or any other similar substance with sufficient germicidal efficacy to airborne pathogens, such as, for instance, hydrogen peroxide. For instance, one embodiment of the present invention may utilize a first compound comprised of triethylene glycol, due to the chemical properties of same. Specifically, because triethylene glycol has a low vapor pressure, low toxicity, high hygroscopicity, and sufficient germicidal properties, triethylene glycol is one such compound disposed to create an aerial germicide with a fluid dispersion device.

Yet, as previously detailed, because of the hygroscopic nature of triethylene glycol, such an operative fluid may yet include a second compound, such as deionized water, which may be disposed to prevent the triethylene glycol from water uptake from the surrounding ambient air remaining in the headspace of the fluid reservoir in which the operative fluid is held. Specifically, by forming a mixture of the triethylene glycol and water, the operative fluid may take on the composition of an ideal liquid as a result of the miscibility of triethylene glycol in water.

Alternatively put, due to the soluble nature of triethylene glycol in water, each liquid in the operative fluid may fully and completely mix together, thereby forming a homogenous solution, while simultaneously maintaining certain properties thereof. For instance, in any given liquid certain molecules will have a tendency to escape the intermolecular forces holding them within the liquid and vaporize at any particular temperature. However, because triethylene glycol is entirely miscible in water, the mixture of each liquid will result in an ideal liquid, wherein the tendency of the different sets of molecules to escape from the liquid and into a vapor form will remain unchanged.

Accordingly, due to the mixture of triethylene glycol and water, the vapor pressure of the operative fluid may be effectively changed to dispose the operative fluid in dynamic equilibrium with the ambient air surrounding same, as the water molecules may simply interchange with the ambient water disposed in the ambient air surrounding such mixture. As may be understood, the term dynamic equilibrium, as used herein, refers to the propensity for the composition of the mixture of the operative fluid to remain unchanged over time. In this manner, the uptake of the water molecules in the ambient air by the triethylene glycol may be effectively reduced or altogether eliminated. Such principle may likewise hold true for embodiments comprising alternative chemical compositions of the mixture which forms the operative fluid. Although other compositions for such an operative fluid are envisioned herein, at least one embodiment of the present invention may comprise an operative fluid formed from a mixture comprising a first percentage of the first compound, and a second percentage of the second compound. More specifically, at least one embodiment of the present invention may comprise a first percentage equal to approximately eighty percent by weight of triethylene glycol and a second percentage equal to approximately twenty percent by weight of water.

Alternatively, other embodiments of the present invention may comprise: (a) any combination wherein a first percentage is within the range of approximately fifty percent to approximately ninety-five percent by weight of triethylene glycol and wherein a second percentage is within the range of approximately five percent to approximately fifty percent by weight of water; (b) a first percentage equal to approximately fifty percent by weight of triethylene glycol and a second percentage equal to approximately fifty percent by weight of water; or (c) a first percentage equal to approximately ninety-five percent by weight of triethylene glycol and a second percentage equal to approximately five percent by weight of water. As will be discussed in greater detail hereafter, such differences in ranges for the first percentage of the first compound and the second percentage of the second compound may be configured so as to address alternative operating conditions, such as atmospheric pressure, in which the applicable fluid dispersion device is operating. As may be understood by one of skill in the art, the term approximately, or any other like terms including, without limitation, “substantially equal,” as used herein is meant to refer only to those differences in tolerances which may result in the formation of such an operative fluid. Likewise, as used herein, the terms first percentage and second percentage, as well as any other terms referring to a percentage of a compound, are made with specific reference to the weight percentage of such compound in relation to the overall weight of the operative fluid.

Further, the operative fluid of at least one embodiment of the present invention may include yet an additional component therein, namely a third compound. Such a third compound may comprise, for instance, an essential oil or other like compound. Such a third compound may be disposed to assist in the germicidal effect of the operative fluid, or alternatively, provide an olfactory signal to those persons situated in the surrounding environment that the fluid dispersion device is operating, or both. For instance, in one embodiment of the present invention wherein the third compound comprises an essential oil, such a third compound may comprise thymol, for both its pleasant aromatic odor and strong antiseptic properties. Likewise, an alternative embodiment of the present invention wherein the third compound comprises an essential oil may utilize eucalyptus oil for such a third compound, due to its similar fragrance and antiseptic properties. Further, in yet an additional embodiment of the present invention wherein the third compound comprises an essential oil, such a third compound may instead comprise tea tree oil, which likewise has certain aromatic properties. As may be understood, such a third compound may comprise any combination of the foregoing and/or additional compounds, whether essential oils or otherwise, provided such third compound does not cause any safety risks to humans in direct exposure to the vapor and/or particles thereof.

In such embodiments wherein a third compound is incorporated into the operative fluid, it may be understood the previously detailed chemical composition may be altered. Specifically, the amount of either the first compound, the second compound, or both the first and second compounds may be altered or otherwise adjusted to compensate for the presence of the third compound in the operative fluid. As may be understood, alternative compositions of the operative fluid are likewise envisioned in the present invention, provided such composition operates to maintain the dynamic equilibrium between the operative fluid and the surrounding ambient air such that the uptake of ambient water by the first compound therein is sufficiently reduced or otherwise eliminated, while simultaneously maintaining the efficacy of such first compound as an aerial germicide.

As may be understood, in order to effectively and efficiently provide an aerial sanitization in accordance with the present invention, it may be necessary to continuously and/or periodically administer the aerial sanitizer formed from the disclosure herein to the applicable environment. Accordingly, in at least one embodiment of the present invention, the previously discussed fluid dispersion device may be structured so as to administer such an aerial sanitizer at predetermined intervals. Such predetermined intervals may comprise for instance, certain time intervals and/or certain amounts of the aerial sanitizer. Such predetermined intervals may be effectuated by an interval component such as, for instance, a timer or other like component disposed to effectively administer the aerial sanitizer when specified. Moreover, such an interval component may be disposed to, for instance, determine the proper time intervals for the application of the fluid dispersion in the surrounding airspace upon, for instance, the user input of certain information which may include, without limitation, the volume of the applicable surrounding airspace, or other variables such as the atmospheric pressure of the surrounding airspace and/or the relative humidity of same.

For instance, as may be understood, the amount of aerial sanitizer which must be dispersed into a given environment, or alternatively the composition of the operative fluid itself, may depend upon, for instance, the volume of such environment. Likewise, where a given environment is disposed at areas of different pressure, such as, for example, environments disposed at higher altitudes, the requisite amount of aerial sanitizer needed for such an environment may change as well. Other similar factors impacting the aforementioned predetermined intervals in which the fluid dispersion need be applied to a surrounding airspace comprise, for instance, the relative humidity, temperature, and rate of ventilation therein. All such factors may be accounted for in determining the amount and/or timing of the application of such fluid dispersion into the surrounding airspace. Accordingly, any such embodiment incorporating the aforementioned ability to administer the aerial sanitizer at predetermined intervals may be so disposed to ensure the environment remains saturated with the aerial sanitizer, thereby ensuring the environment is effectively sanitized.

These and other objects, features, and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a front view of a fluid dispersion device, to be used in accordance with at least one embodiment of the present invention.

FIG. 1A is a front view of a fluid dispersion device, to be used in accordance with at least one embodiment of the present invention.

FIG. 1B is a front view of a fluid dispersion device, to be used in accordance with at least one embodiment of the present invention.

FIG. 2 is a schematic table of at least one embodiment of an operative fluid, in accordance with at least one embodiment of the present invention.

FIG. 2A is a schematic table of at least one embodiment of an operative fluid, in accordance with at least one embodiment of the present invention.

FIG. 2B is a schematic table of at least one embodiment of an operative fluid, in accordance with at least one embodiment of the present invention.

FIG. 2C is a schematic table of at least one embodiment of an operative fluid, in accordance with at least one embodiment of the present invention.

FIG. 3 is a schematic table of alternative embodiments for a third compound in accordance with the present invention.

FIG. 4 is a schematic table of an operative fluid in accordance with at least one embodiment of the present invention.

FIG. 5 is a schematic representation of the top surface of a fluid dispersion device in accordance with at least one embodiment of the present invention.

Like reference numerals refer to like parts throughout the several views of the drawings.

### DETAILED DESCRIPTION OF THE PREFERRED EMBODIMENT

The present invention is directed to a chemical composition, the product formed therefrom, and a method for producing same. Specifically, the present invention is directed to an aerial germicide, or otherwise an aerial disinfectant and/or a fluid dispersion, designed to be utilized in accordance with a fluid dispersion device.

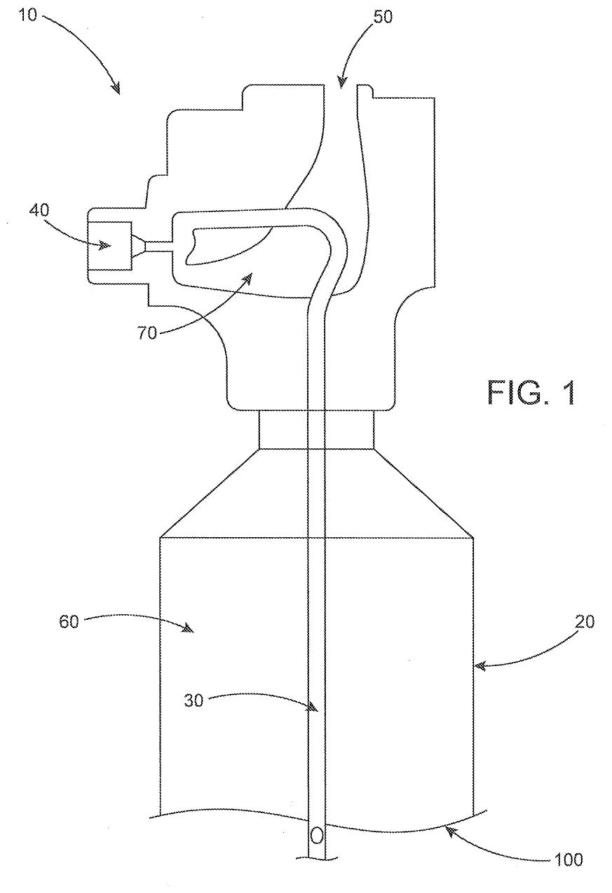

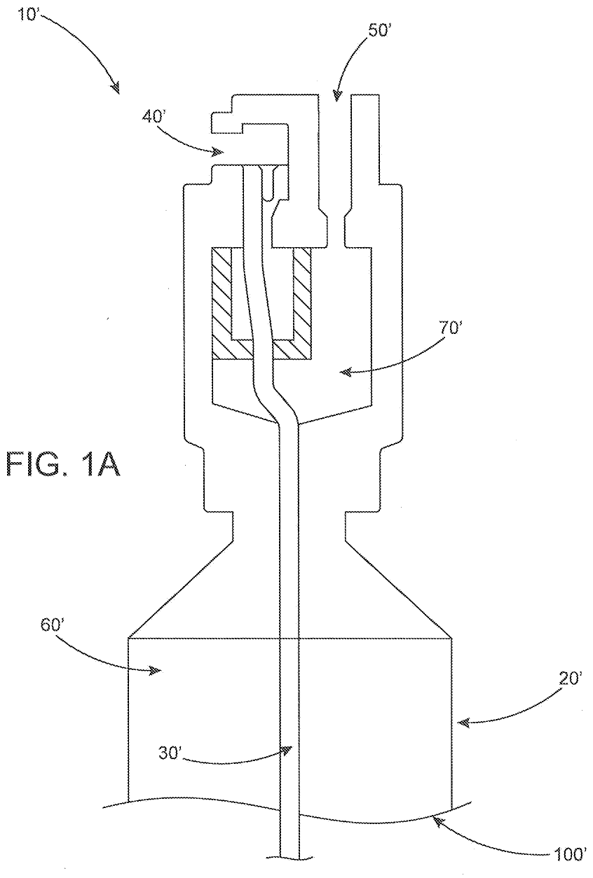

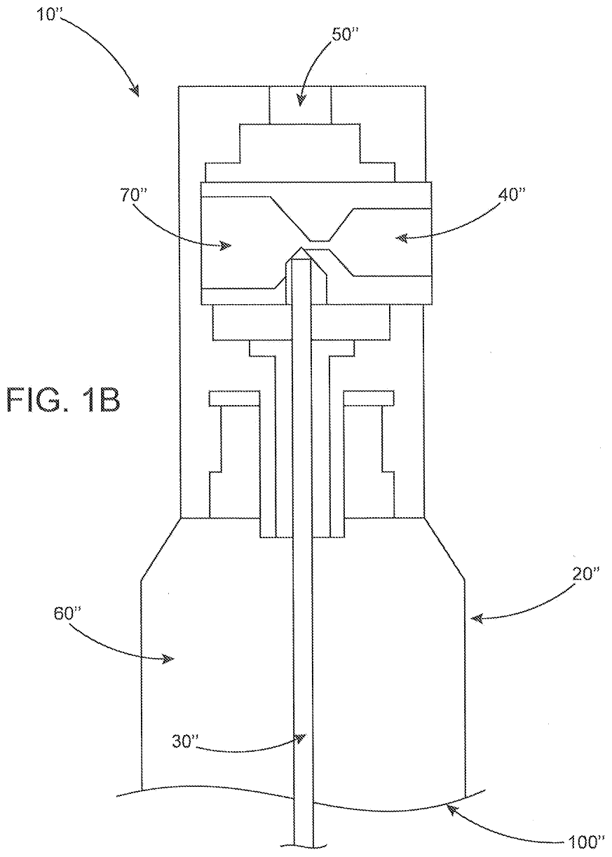

For example, one embodiment of such a fluid dispersion device may be seen with reference to FIG. 1. Such an embodiment may comprise, without limitation, at least some components present in the fluid dispersion assembly disclosed in U.S. Pat. No. 10,583,449. Specifically, such a fluid dispersion device 10 may comprise at least a reservoir 20 for containing both an operative fluid 100 and ambient air 60. Such a reservoir 20 may be connected to a pressurized air component 40 and a reaction chamber 70 through an elongated tube 30 disposed in fluid communication there between, such that the operative fluid 100 may be withdrawn from the reservoir 20 upon the application of pressurized air thereon according to the venturi effect. Upon such a withdrawal of the operative fluid 100, the pressurized air may then disperse same into uniform droplets, to be mixed with the pressurized air, thereby forming a fluid dispersion comprising an aerial germicide.

Upon such formation, the aerial germicide may then be expelled from such a fluid dispersion device 10 via an outlet 50, thereby disseminating the aerial germicide into the surrounding airspace. In so doing, such a fluid dispersion device 10 may effectively create and disseminate an aerial germicide comprising a fluid dispersion comprised of a gaseous medium with miniscule liquid particles comprising uniform droplets of the operative fluid 100 disposed therein. Such uniform droplets may comprise a size of approximately 1 micrometer or less, thereby allowing such miniscule liquid particles to effectively condense on airborne microorganisms so a germicidal concentration of the operative fluid 100 accumulates thereon, thereby effectuating a lethal response to any airborne pathogens present in the airspace surrounding the fluid dispersion device 10.

Alternative embodiments of a fluid dispersion device 10 may comprise certain alternative components, structures, systems, and/or methods disposed to similarly effectuate the dissemination of such an aerial germicide into the airspace surrounding the fluid dispersion device 10. For example, such alternative embodiments may comprise, without limitation, a jet nebulizer, a concentric tube pneumatic nebulizer, or a diffuser. Accordingly, such alternative embodiments may be disposed to create an aerosol compound comprising a gaseous medium with miniscule liquid particles disposed therein. Such miniscule liquid particles may likewise comprise, in at least some embodiments, a size of approximately 1 micrometer or less, however, alternative sizes are envisioned herein provided such liquid particles are so disposed to effectively condense on airborne microorganisms, as previously stated.

For instance, an alternative embodiment of a fluid dispersion device 10′, as seen in FIG. 1A, may comprise at least a reservoir 20′ for containing both an operative fluid 100′ and ambient air 60′. Such a reservoir 20′ may be connected to a pressurized air component 40′ and a reaction chamber 70′ through an elongated tube 30′ disposed in fluid communication there between, such that the operative fluid 100′ may be withdrawn from the reservoir 20′ upon the application of pressurized air. Upon such a withdrawal of the operative fluid 100′, the formed fluid dispersion comprising an aerial germicide may be expelled from such a fluid dispersion device 10′ via an outlet 50′, thereby disseminating the aerial germicide into the surrounding airspace. In another alternative embodiment of a fluid dispersion device 10″, as seen in FIG. 1B, an operative fluid 100″ and ambient air 60″ may be contained within a reservoir 20″, which in turn may be connected to a pressurized air component 40″ and reaction chamber 70″ via an elongated tube 30″. Pressurized air may be used in conjunction with such a fluid dispersion device 10″, which may withdraw the operative fluid 100″ from the reservoir 20″ to form a fluid dispersion comprising an aerial germicide. The fluid dispersion may then be expelled from such a fluid dispersion device 10″ via an outlet 50″. Accordingly, as may be seen, a variety of fluid dispersion devices may be used in connection with the present invention, provided such fluid dispersion devices effectively create the aerial germicide discussed herein.

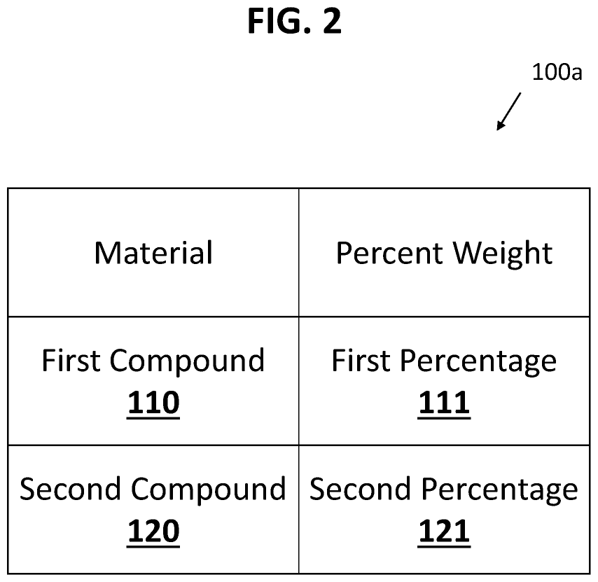

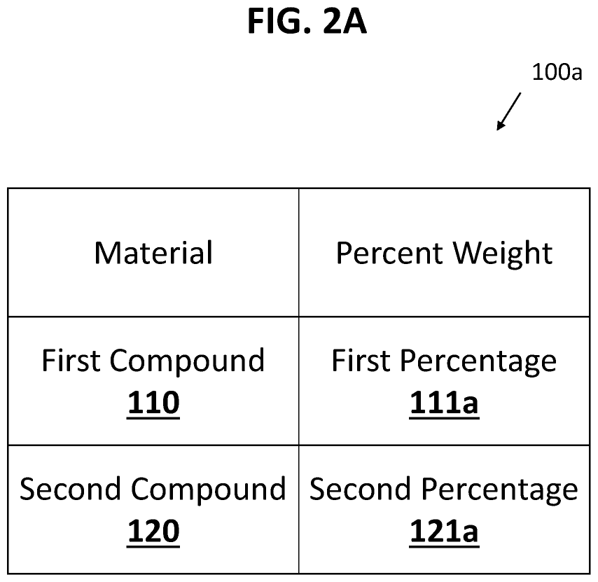

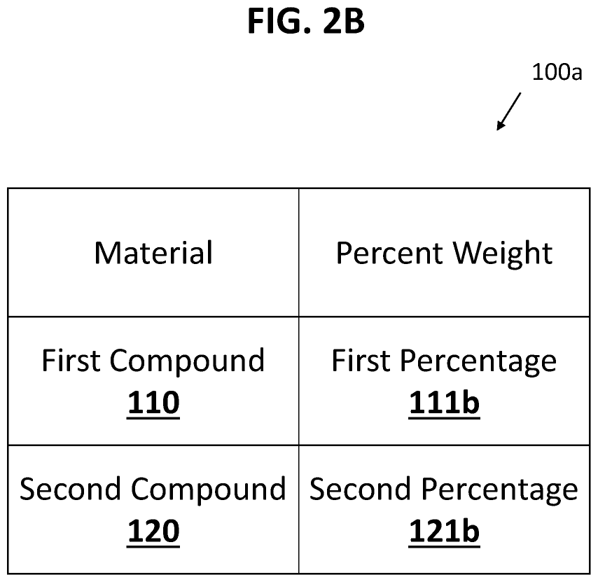

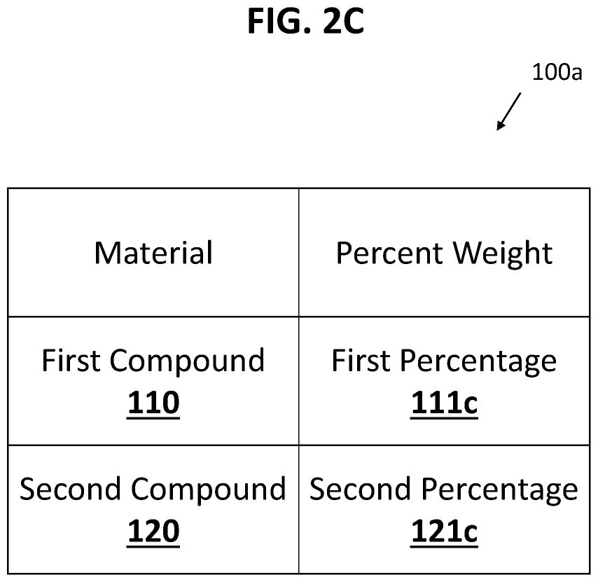

Generally speaking, the aforementioned fluid dispersion formed through such a fluid dispersion device 10, whether in the embodiment depicted in FIGS. 1-1B or otherwise, may comprise at least the operative fluid 100 disposed in the reservoir 20. As such, alternative embodiments may comprise alternative chemical compositions of such an operative fluid 100. For instance, in the embodiment depicted in FIG. 2, such an operative fluid 100 may comprise a first compound 110 and a second compound 120, wherein the first compound 110 and the second compound 120 comprise a first percentage 111 and a second percentage 121 by weight of the operative fluid 100, respectively. As previously stated, as used herein, the terms first percentage 111 and second percentage 121, as well as any other terms referring to a percentage of a compound, are made with specific reference to the weight percentage of such compound in relation to the overall weight of the operative fluid 100.

More specifically, the operative fluid 100 in the embodiment depicted in FIG. 2 may comprise a first compound 110. Such a first compound 110 may comprise, for instance, triethylene glycol (CAS #112-27-6), due to its low vapor pressure, high hygroscopicity, and sufficient germicidal properties. Such a first compound 110 may comprise alternative compounds, provided such compounds exhibit chemical properties similar to those enunciated herein for triethylene glycol. Specifically, alternative embodiments of such a first compound 110 should be chemically disposed to create an effective aerial germicide with such a fluid dispersion device 10. For instance, such a first compound 110 may instead comprise chemical compositions such as other glycols, such as propylene glycol, or other chemical compositions not comprising glycols, such as hydrogen peroxide. In such embodiments, it may be understood the composition of the operative fluid 100 may vary in accordance with the chemical properties of such first compound 110, both as to the chemical composition of any alternative compounds disposed therein, and the weight percentages of same.

As previously detailed, because of the hygroscopic nature of such a first compound 110, an operative fluid 100 in accordance with at least one embodiment of the present invention may comprise a second compound 120, as may be seen with reference to FIG. 2. Such a second compound 120 may comprise, for instance, deionized water (CAS# 7732-18-5). The inclusion of such a second compound 120 may be operatively inclined to prevent unintended water uptake by the first compound 110 from the ambient air 60 likewise disposed within the reservoir 20 of the fluid dispersion device 10. Specifically, by creating a mixture of the first compound 110 and the second compound 120, the operative fluid 100a may take on a composition of an ideal liquid as a result of the miscibility of the first compound 110 in the second compound 120, and thereby reach a level of dynamic equilibrium with the ambient air 60.

For example, in embodiments wherein the first compound 110 comprises triethylene glycol and the second compound 120 comprises deionized water, it may be understood the first compound 110 and the second compound 120 may form an operative fluid 100a comprising a homogeneous mixture when same are added together. Alternatively put, due to the soluble nature of triethylene glycol in water, each liquid in the operative fluid 100a may fully and completely mix together, thereby forming a homogenous solution, while simultaneously maintaining certain properties thereof. For instance, in any given ideal liquid, certain molecules will have a tendency to escape the intermolecular forces holding them within the liquid and vaporize at any particular temperature. Thus, because triethylene glycol is entirely miscible in water, the mixture of each liquid will result in an ideal liquid, wherein the tendency of the different sets of molecules to escape from the liquid and into a vapor form will remain unchanged.

Accordingly, due to the mixture of triethylene glycol and water, the vapor pressure of the operative fluid 100a may be effectively changed to dispose the operative fluid 100a in dynamic equilibrium with the ambient air 60 surrounding same. In this manner, the uptake of the water molecules in the ambient air 60 by the first compound 110 may be effectively reduced.

In accordance therewith, it may be understood alternative embodiments of the present invention may comprise different chemical compositions of the mixture which forms the operative fluid 100a. For instance, in the embodiment depicted in FIG. 2, the first percentage 111 may comprise an amount of the first compound 110 of approximately eighty percent of the weight of the operative fluid 100a. Accordingly, the second percentage 121 may comprise an amount of the second compound 120 of approximately twenty percent by weight of the operative fluid 100a. As may be understood by one of skill in the art, the term approximately as used herein is meant to refer only to those differences in tolerances which may result in the formation of such an operative fluid 100a. Moreover, it may be understood alternative compositions of a first compound 110 and a second compound 120 are envisioned herein, such as, for instance, those instances dictated by the volume of the reservoir 20, those embodiments utilizing alternative chemical compositions for the first compound 110 and/or the second compound 120, or those embodiments wherein the present invention is disposed in regions of alternative ranges of atmospheric pressure, relative humidity, and/or temperatures.

In an alternative embodiment of the present invention, depicted in FIG. 2C, the first percentage 111c may comprise an amount of the first compound 110 within the range of approximately fifty percent to approximately ninety-five percent of the weight of the operative fluid 100a. Accordingly, the second percentage 121c may comprise an amount of the second compound 120 within the range of approximately five percent to approximately fifty percent by weight of the operative fluid 100a. In another embodiment of the present invention, depicted in FIG. 2A, the first percentage 111a may comprise an amount of the first compound 110 of approximately fifty percent of the weight of the operative fluid 100a. Accordingly, the second percentage 121a may comprise an amount of the second compound 120 of approximately fifty percent by weight of the operative fluid 100a. In yet another alternative embodiment of the present invention, depicted in FIG. 2B, the first percentage 111b may comprise an amount of the first compound 110 of approximately ninety-five percent of the weight of the operative fluid 100a. Accordingly, the second percentage 121b may comprise an amount of the second compound 120 of approximately five percent by weight of the operative fluid 100a. As may be understood by one of skill in the art, the term approximately as used herein is meant to refer only to those differences in tolerances which may result in the formation of such an operative fluid 100a. Moreover, it may be understood alternative compositions of a first compound 110 and a second compound 120 are envisioned herein, such as, for instance, those instances dictated by the volume of the reservoir 20, those embodiments utilizing alternative chemical compositions for the first compound 110 and/or the second compound 120, or those embodiments wherein the present invention is disposed in regions of alternative ranges of atmospheric pressure, relative humidity, and/or temperatures.

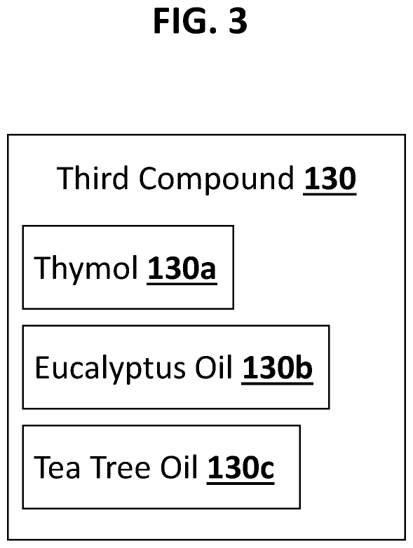

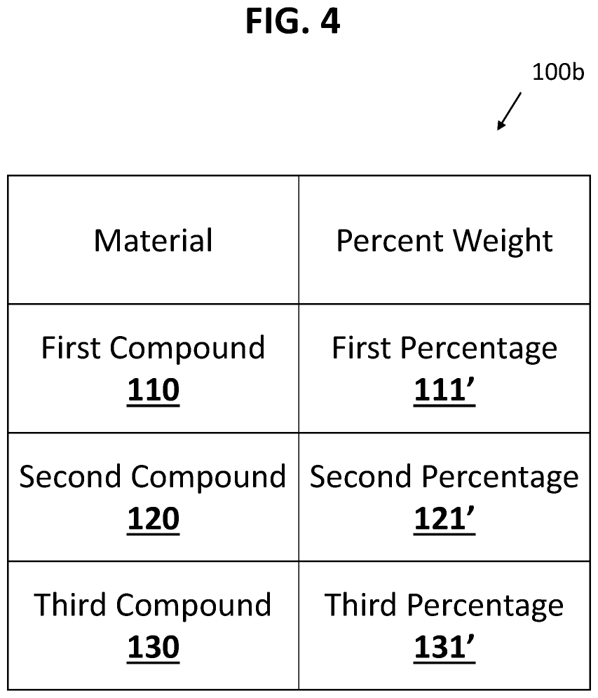

In yet additional embodiments of the present invention, such as the one depicted in FIG. 4, the operative fluid 100b may additionally comprise a third compound 130. Such a third compound 130 may comprise, as may be seen in FIG. 3, an essential oil including, without limitation, thymol 130a, eucalyptus oil 130b, and/or tea tree oil 130c. Of course, alternative embodiments of the present invention may instead comprise a third compound 130 comprising a combination of the aforementioned essential oils, alternative essential oils, and/or other compounds not recited herein. Such a third compound 130 may be inclined to assist in the germicidal effect of the operative fluid 100b and/or provide an olfactory signal to those persons situated in the surrounding environment of the presence of the aerial germicide therein.

For instance, where the third compound 130 comprises thymol 130a, it may be understood the addition thereof may be predisposed to utilize both the pleasant aromatic odor and strong antiseptic properties of same Likewise, eucalyptus oil 130b may be utilized as the third compound 130 due to both its fragrance and its antiseptic properties. In a similar vein, the third compound 130 may also comprise tea tree oil 130c for its aromatic properties. Accordingly, as may be understood, such a third compound 130 may likewise comprise a combination of same, alternative essential oils, or otherwise, provided such a third compound 130 provides similar benefits, whether aromatic, antiseptic, or otherwise, and does not cause any safety risks to humans in direct exposure to the vapor and/or liquid particles thereof.

As may be understood, in embodiments wherein the operative fluid 100b comprises a third compound 130, the chemical composition thereof may be adjusted to reflect the inclusion of same. The amount of either the first compound 110, the second compound 120, or both the first compound 110 and second compound 120 may be altered or otherwise adjusted to compensate for the presence of the third compound 130 in the operative fluid. Specifically, as depicted in FIG. 4, such an embodiment may comprise a first percentage 111′ in the range of approximately fifty percent to approximately ninety-five percent by weight of the operative fluid 100b for the first compound 110; a second percentage 121′ in the range of approximately five percent to approximately fifty percent by weight of the operative fluid 100b for the second compound 120; and a third percentage 131′ of at least 0.1 percent by weight of the operative fluid 100b for the third compound 130, as determined by the removal of an amount of either the first compound 110, the second compound 120, or both the first compound 110 and second compound 120. For instance, such a third percentage 131′ could comprise a range of approximately 0.1% to 1%, or in some embodiments, an even greater amount. As may be understood by one of skill in the art, the term approximately as used herein is meant to refer only to those differences in tolerances which may result in the formation of such an operative fluid 100b. Alternative embodiments may comprise alternative compositions of the operative fluid 100b, provided such composition operates to maintain the aforementioned dynamic equilibrium between the operative fluid 100b and the surrounding ambient air 60 while simultaneously providing the desired germicidal effect.

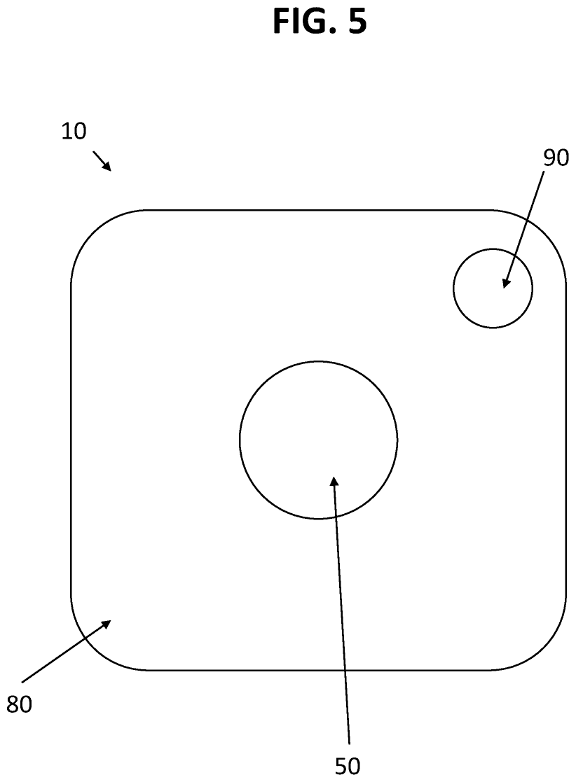

As previously discussed, at least some embodiments of the present invention may be devised such that the operative fluid 100 may be withdrawn from the reservoir 20 and formed into the aerial germicide continuously and/or periodically. Accordingly, at least one embodiment of the present invention, such as the one depicted in FIG. 5 may comprise an interval component 90, such as a timer or other like component, disposed on the top portion 80, or any other alternative location, of the fluid dispersion device 10, wherein such interval component 90 is disposed to effectively administer the aerial sanitizer when specified. Such an application of the aerial sanitizer may occur at, for instance, predetermined intervals set by a user.

For instance, as may be understood, the amount of aerial sanitizer which must be dispersed into a given environment may depend upon, for instance, the volume, relative humidity, temperature, and/or rate of air flow of such an environment Likewise, where a given environment is disposed at areas of different atmospheric pressure, such as, for example, environments disposed at higher altitudes, it may be understood the requisite amount of aerial sanitizer needed for such an environment may change as well. Accordingly, any such embodiment incorporating the aforementioned ability to administer the aerial sanitizer at predetermined intervals may be so disposed to ensure the environment remains saturated with the aerial sanitizer, whether such disposition occurs as a result of a user adjusting such predetermined intervals or otherwise. Likewise, it is envisioned herein, such an interval component, such as a timer, for applying the aerial sanitizer at predetermined intervals may likewise be disposed to automatically adjust such predetermined intervals by, for example, measuring for the atmospheric pressure at which the fluid dispersion device 10 is situated, or otherwise interlinking with a device, such as, for instance, a smartphone, which may be disposed to provide any and/or all such pertinent information to the fluid dispersion device 10, for the calculation of sufficient predetermined intervals configured to ensure the surrounding airspace remains saturated with the fluid dispersion at all times.

The present invention seeks to avoid many of the problems experienced when using traditional aerial sanitization systems. Reliance on volatile hydrocarbon propellants may be reduced by instead opting to apply pressurized air to an aerosol formulation, which is not volatile and does not carry certain occupational and/or health risks. By applying pressurized air, the particle size within the aerosol formulation may be reduced, which then may lead to a gaseous aerosol formulation that is able to reach all areas of a room and disinfect same. Further, in embodiments of the present invention that may contain a second compound 120 in addition to triethylene glycol within an operative fluid 100, the disposition of said second compound 120 may prevent the triethylene glycol from water uptake from the surrounding ambient air 60, thus reducing the tendency of the operative fluid 100 to quickly condense on surfaces and simultaneously increasing the efficacy of aerial sanitization. Moreover, by dispersing the triethylene glycol at the appropriate droplet size and in appropriate intervals, the surrounding airspace may remain saturated with an aerial germicide whose droplets may be small enough to bind to airborne pathogens and eliminate them. Finally, in embodiments of the present invention that may contain a third compound 130 within an operative fluid 100, the dispersion of such an operative fluid 100 may produce sensory signals which may indicate the level of cleanliness of the surrounding airspace.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. An operative fluid to be used for the creation of an aerial germicide, said operative fluid comprising:
a first percentage of a first compound, said first compound comprising triethylene glycol;
a second percentage of a second compound, said second compound comprising deionized water; and
the mixture of said first compound and said second compound devised in dynamic equilibrium with the surrounding ambient air.

2. The operative fluid of claim 1, wherein the concentration of said first percentage is between 50 percent per weight to 95 percent per weight of said operative fluid and the concentration of said second percentage is between 50 percent per weight to 5 percent per weight of said operative fluid.

3. The operative fluid of claim 2, wherein the concentration of said first percentage is substantially equal to 80 percent per weight of said operative fluid and the concentration of said second percentage is substantially equal to 20 percent per weight of said operative fluid.

4. The operative fluid of claim 2, wherein said operative fluid further comprises a third component, said third component comprising an essential oil.

5. The operative fluid of claim 4, wherein the concentration of said third compound is substantially equal to a range comprising at least 0.1 percent per weight.

6. The operative fluid of claim 4, wherein said essential oil is selected from the group consisting of thymol, tea tree oil, and eucalyptus oil.

7. An aerial germicide formed by a fluid dispersion device, said aerial germicide comprising:
a fluid dispersion comprising an operative fluid and the application of pressurized air thereon;
said operative fluid comprising a first compound, at a composition comprising a first percentage, and a second compound, at a composition comprising a second percentage;
said first compound comprising triethylene glycol; and
said second compound comprising deionized water.

8. The aerial germicide of claim 7, wherein said first percentage is between 50 percent per weight to 95 percent per weight of said operative fluid and said second percentage is between 50 percent per weight to 5 percent per weight of said operative fluid.

9. The aerial germicide of claim 7 wherein said operative fluid further comprises a third compound.

10. The aerial germicide of claim 7, wherein said third compound comprises an essential oil.

11. The aerial germicide of claim 10, wherein said essential oil is selected from the group consisting of thymol, eucalyptus oil, and tea tree oil.

12. The aerial germicide of claim 10, wherein the composition of said second compound is a second percentage.

13. The aerial germicide of claim 12, wherein said second percentage is substantially equal to a range comprising 19 percent to 19.9 percent of the weight of said operative fluid.

14. The aerial germicide of claim 7, wherein said fluid dispersion device comprises an interval component operatively structured to dispense said aerial germicide from said fluid dispersion device at predetermined intervals.

15. An aerial germicide formed by a plurality of steps comprising:
withdrawing an operative fluid disposed within a reservoir through an elongated tube, the operative fluid comprising a first compound and a second compound;
applying pressurized air to the operative fluid to create a dispersion of the operative fluid;
mixing the pressurized air with the dispersion of the operative fluid; and
disseminating the combination of the pressurized air and the dispersion of the operative fluid through an outlet to a surrounding airspace of a fluid dispersion device.

16. The aerial germicide of claim 15, wherein the first compound comprises triethylene glycol.

17. The aerial germicide of claim 15, wherein the second compound comprises deionized water.

18. The aerial germicide of claim 15, wherein the operative fluid further comprises a first percentage of the first compound, and a second percentage of the second compound.

19. The aerial germicide of claim 18, wherein the first percentage of the first compound is within the range of approximately fifty percent to approximately ninety-five percent of the weight of the operative fluid

20. The aerial germicide of claim 18, wherein the second percentage of the second compound is within the range of approximately five percent to approximately fifty percent by weight of the operative fluid.

21. The aerial germicide of claim 15, wherein the operative fluid further comprises a third compound.

22. The aerial germicide of claim 21, wherein the third compound is an essential oil.

23. The aerial germicide of claim 21, wherein the operative fluid further comprises a third percentage of the third compound, the third percentage comprising a range of approximately 0.1 percent by weight of the operative fluid to 1 percent by weight of the operative fluid.
